(19) United States
(12) Patent Application Publication
(10) Pub. No.: US 2024/0342333 A1
(43) Pub. Date: Oct. 17, 2024

(54) METHODS FOR MONITORING AND TRACKING STERILE PROCESSING OF SURGICAL INSTRUMENTS

(71) Applicant: Bedrock Surgical, Inc, Palo Alto, CA (US)

(72) Inventors: Gregory Faris, Menlo Park, CA (US); Yingdi Liu, Santa Clara, CA (US); Richard Hill, Campbell, CA (US); David Stoker, Belmont, CA (US); Erik Matlin, San Jose, CA (US)

(73) Assignee: Bedrock Surgical, Inc, Palo Alto, CA (US)

(21) Appl. No.: 18/682,914

(22) PCT Filed: Aug. 11, 2022
(86) PCT No.: PCT/US22/40135

§ 371 Date: Feb. 11, 2024

Related U.S. Application Data
(60) Provisional application No. 63/232,100, filed on Aug. 11, 2021.

(51) Int. Cl. A61L 2/28; G16H 40/40

(52) U.S. Cl. CPC A61L 2/28; G16H 40/40; A61L 2202/24

(57) ABSTRACT

The system and methods described herein are directed to monitoring and tracking surgical instruments through sterilization processing. Imaging methods described are performed based on a variety of needs such as to monitor damage, wear, and biomass. Also described are how automation, vision augmentation, and data management systems can be incorporated into the system to facilitate monitoring and tracking. The system and methods described here will use specialized optical imaging methods together with automation and image analysis methods to perform inspection and tracking of surgical instruments faster, more accurately, and less expensively than using existing methods. The new imaging methods disclosed include ultraviolet fluorescence, polarization imaging, oblique illumination, and extensive light source in reflection. Further, the methods include using multiple imaging methods and imaging methods in combination with automation and imaging processing for assessing surgical instruments. By implementing the disclosed methods, more favorable surgical outcomes will likely result.

### CROSS-REFERENCES

This application claims the benefit of U.S. Provisional Application No. 63/232,100, filed Aug. 11, 2021.

### BACKGROUND

This technology relates generally to sterile processing of surgical instruments and, more particularly, to methods of monitoring, tracking and maintenance of proper safety and efficacy protocols for surgical instruments through consistency of detection of abnormalities when the surgical instruments are processed via a consistent repeatable process. U.S. Provisional Application No. 63/232,100, filed Aug. 11, 2021, the entire disclosure of which, except for any definitions, disclaimers, disavowals, and inconsistencies, is incorporated herein by reference.

### SUMMARY

In sterile processing departments, it is of utmost importance that instruments that are being sent to operating rooms are sterile and free of hidden defects. Unfortunately, human visual inspection often misses both physical contamination and instrument damage.

In developing a mechanism to detect issues before they are placed into service it has become clear that there is a need for a repeatable process that allows detection with a high confidence level.

There exists a need for process that consistently catches devices that need to be removed from service.

When deciding about design choices with respect to a system in accordance with the present invention, simplicity is a preferable design choice that is disclosed herein.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of embodiments in accordance with the present invention are apparent in the following detailed description and claims.

### DESCRIPTION OF THE FIGURES

The patent or application file contains at least one image executed in color. Copies of this patent or patent application publication with color drawing(s) will be provided by the Office upon request and payment of the necessary fee.

Various example embodiments can be more completely understood in consideration of the following detailed description in connection with the accompanying drawings, in which:

FIG. 1 shows a diagram of one embodiment of an imaging system for fluorescent biomass detection.

FIG. 2 shows a series of excitation and emission spectra for protein/bioburden, detergent, rinse, and lubricant. Excitation spectra are on the left of each pair of spectra and emission spectra are on the right.

FIG. 3 shows images taken of a surgical tool with ambient light only (left) compared with an extensive light source in reflection (right).

FIG. 4 shows another set of images taken of a surgical tool with ambient light only (left) compared with polarization imaging (right).

FIG. 5 shows one possible arrangement of multiple sensors around a tool.

FIG. 6 shows one possible arrangement of a single sensor around a tool.

### DETAILED DESCRIPTION

This technology relates generally to surgical instruments and, more particularly, to apparatus' for and methods of assessing the safety, efficacy and continued suitability of surgical instruments as part of the sterile processing protocol.

This disclosure describes inventive concepts with reference to specific examples. However, the intent is to cover all modifications, equivalents, and alternatives of the inventive concepts that are consistent with this disclosure. It will be apparent, however, to one of ordinary skill in the art that the present approach can be practiced without these specific details. Thus, the specific details set forth are merely exemplary, and is not intended to limit what is presently disclosed. The features implemented in one embodiment may be implemented in another embodiment where logically possible. The specific details can be varied from and still be contemplated to be within the spirit and scope of what is being disclosed.

The system and methods described herein are directed to monitoring and tracking surgical instruments through sterilization processing. Imaging methods described are performed based on a variety of needs such as to monitor damage, wear, and biomass. Also described are how automation, vision augmentation, and data management systems can be incorporated into the system to facilitate monitoring and tracking.

Currently, evaluation is primarily done with visual inspection. Visual inspections often miss important details that could eventually lead to instrument failure during important procedures. Other currently available systems for finding biomass are slow and expensive. The system and methods described here will use specialized optical imaging methods together with automation and image analysis method to perform inspection and tracking of surgical instruments faster, more accurately, and less expensively than using existing methods.

The new imaging methods disclosed include ultraviolet fluorescence, polarization imaging, oblique illumination, and extensive light source in reflection. Further, the methods include using multiple imaging methods and imaging methods in combination with automation and imaging processing for assessing surgical instruments. By implementing the disclosed methods, more favorable surgical outcomes will likely result.

Multimodal Imaging System. The multimodal imaging system provides information that affects whether instruments are ready for use or may require additional cleaning, repair, replacement, or removal from service. The primary considerations include instrument damage or wear and contamination such as due to bioburden, or residual cleaning or lubrication agents. Combining multiple imaging modalities provides several advantages including (1) faster measurements when instruments don't need to be repositioned between inspection steps; (2) lower system costs and complexity; and (3) better results—when multiple modalities image the same region, the different contrast mechanisms can be used in a synergistic manner to improve identification of key features. For example, combining conventional visible imaging with UV fluorescence measurements provides context on the location of biomass. This information could be used variously to focus cleaning efforts on a particular tool to meet standards or requirements, to provide feedback on efficacy of current cleaning methods, to enhance training of SPD personnel, or to refine instrument design to minimize accumulation of biomass. When combining different contrast mechanisms such as polarization imaging, oblique illumination imaging, or infrared imaging, the different contrast mechanisms provide different types of information that may be able to detect damage more reliably than a single imaging method or allow better differentiation between different types of damage. For example, combining information from two or more modalities may provide cues that will differentiate between scratches and cracks or determine differences between discoloration and pitting. In this way one can differentiate between what may be cosmetic issues such as scratches or discoloration and significant damage that will require removal of a tool from use such as cracks and pitting. Imaging modalities and their capabilities are described in the following.

Imaging modalities for sensing damage and/or bioburden can include but are not limited to: (a) Visible—damage detection (cracks, pitting), QR code identification, bioburden fluorescence detection; (b) UV—bioburden fluorescence detection; (c) IR—damage detection (cracks, pitting); (d) Imaging detectors are preferred for wide area coverage and rapid inspection, but line or point scanned detectors could also be used; and/or (e) Unpolarized and polarized detectors have utility. Polarized light imaging can be used to highlight damage.

UV fluorescence. Biomass can be measured using the intrinsic fluorescence of amino acids in protein. Three amino acids—tryptophan, tyrosine and phenylalanine—fluoresce due to their aromatic rings. The fluorescence of the tryptophan residues tends to dominate. These are excited near 280 nm with emission peaking near 350 nm. We have demonstrated how this emission can be used to detect bioburden on surgical tools. Other methods exist for fluorescence monitoring of biomass, but these other methods require the use of an additional reagent spray and further require placing a single tool at a time in a box for detection.

One of the challenges in using intrinsic fluorescence for biomass detection is obtaining sufficient light near 280 nm. Until recently, it was difficult to obtain a good light source for this wavelength. Mercury lamps are often used for ultraviolet radiation, but they do not provide the appropriate wavelength of 280 nm. Other sources such as xenon lamps or specialized lasers are expensive. Fortunately, high power LEDs emitting at 280 nm are now available. As an example, some systems offer 280 nm LEDs with maximum powers up to 70 mW. It was shown that this is ample power for imaging biomass.

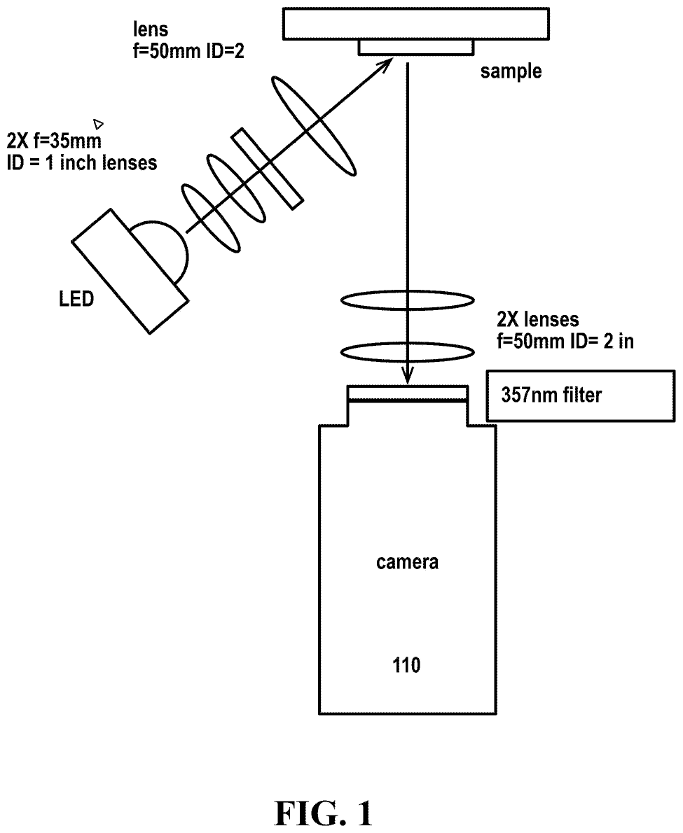

An imaging system used for fluorescence biomass detection is shown in FIG. 1. A high power 280 nm LED is shaped by lenses and illuminates the sample. Filters on the LED and the camera are used to control the excitation spectral band and emission spectral band, as is generally used for fluorescence imaging. These filters prevent direct detection of scattered excitation light from contributing the image, which would otherwise produce excess background light in the image. Because of the short wavelengths of the excitation and emission light, optics with good transmission in the ultraviolet should be used such as fused silica.

An example of this fluorescent imaging method for imaging blood is illustrated below:

As you view the two images above, note that the one on the left is a blood smear on paper taken with ambient light and the one on the right is taken with intrinsic fluorescence.

Images were acquired of a blood smear on a paper towel. The image at left was acquired with ambient light. The blood smear is evident as a discoloration on the towel. A fluorescence image is shown at right. Although the smear is evident in the ambient light image, the contrast is extremely high for the fluorescence image because there is essentially no fluorescence when there is no blood present. The blood smear is very bright whereas there is essentially no signal from the paper towel. This shows the power of properly performed fluorescence imaging where the contrast between the fluorescent substance (blood in this case) and the remaining material can be extremely high.

Above are image of tools is with bovine serum albumin (BSA) as surrogate for biomass. The image at left shows the tools as taken with conventional reflection illumination, in this case taken with light that passes through the emission filter. A second fluorescence image is taken using the 280 nm LED illumination and the ambient illumination turned off. This image is overlaid on the reflection image to create the image at right. The overlay is a useful way to display the biomass detection because it shows the location of the biomass relative to the tool. The fluorescence image was acquired with an integration time of 1 second. Fluorescence images have been acquired with as little as 130 ms exposure time, as shown in the example images of biomass detection on surgical tools are shown below:

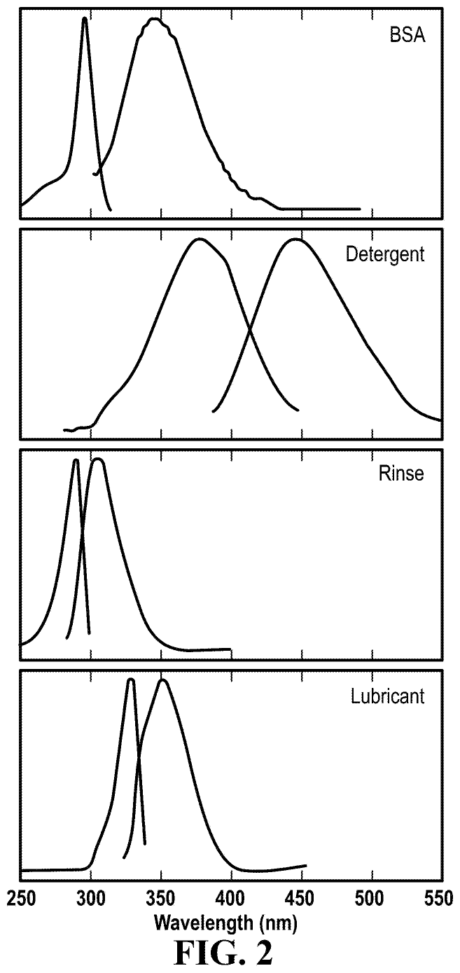

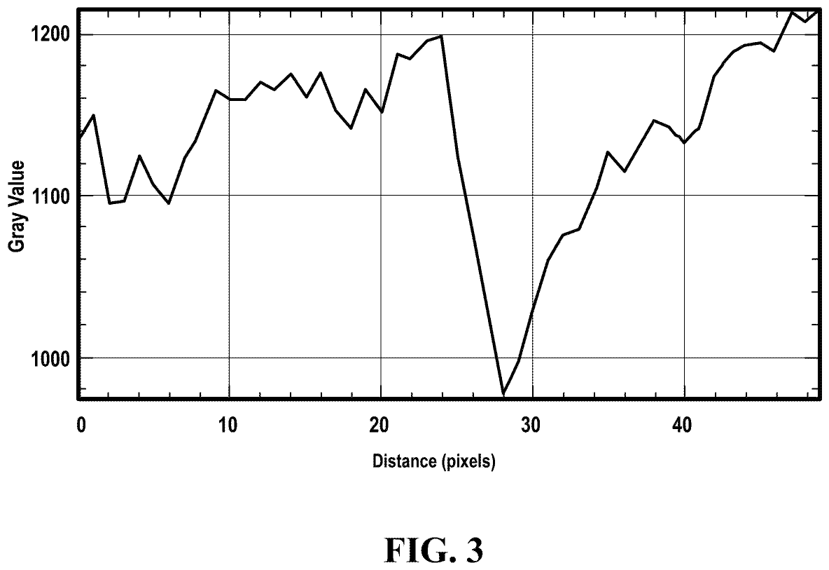

While bioburden is of great significance to surgical tool inspection in the SPD, other materials can also fluoresce, including detergent, rinse, and lubricant. Detection of these is also useful to check that contamination is not left on an instrument. The different materials can be distinguished based on their fluorescence spectral properties. Examples of excitation and emission spectra for protein/bioburden, detergent, rinse, and lubricant are shown in FIG. 2. Bovine serum albumin is used as a surrogate for bioburden/protein. Albumin is the dominant protein in blood. As can be seen from the figure, each type of possible contaminant has unique fluorescence excitation and emission spectra, which may be used to distinguish the type of contaminant. For example, excitation at 280 nm can efficiently excited both protein/BSA and rinse. By monitoring the emission wavelengths of 310 and 350 nm, one can distinguish between protein and rinse depending on which wavelength gives a stronger signal.

Sensor Combinations. The visible and UV imaging methods can be combined effectively because they can share the same camera. Thus, any combination of UV fluorescence, visible/ambient light, visible oblique illumination, visible/polarized imaging, and visible extensive reflection imaging can be combined using the same camera and swapping the light sources. Filters in front of the camera to block excitation light for the UV fluorescence can be selected to also transmit light for the other visible imaging modalities. It is unlikely that all damage and bioburden can be effectively detected with a single detector type. Significant utility can be gained by combining the results from more than one sensor type: (a) Different sensors, similar views, results combined in post processing to provide comparisons; (b) Different sensors, different views; (c) Single, multi-modal sensor capable of more than one detection/measurement technique. This could entail multiple wavelength ranges (e.g., visible and near-IR) or detection techniques (e.g., polarized vs. unpolarized); among other advantages.

Turning specifically to the application of the systems described above, utilizing imaging generally and multiple imaging techniques can give you various data perspectives to ensure that you have a comprehensive picture as to whether an instrument is ready for use, needs to be reprocessed, repaired or taken out of service all together.

Below are images of surgical tools taken with conventional reflected light illumination (left), with fluorescence (center), and superimposed UV intrinsic fluorescence overlaid on reflected light (right).

Extensive light source reflection. The contrast for cracks and pits can also be enhanced using extensive light source in reflection. In this mode, a large uniform illumination is placed behind the camera. In this case, the illumination was achieved by directing a bright light source against a white wall. Because the light source is behind the camera, the image emphasizes reflections from the tool. Light that strikes cracks or pits is reflected less efficiently because of the irregular surfaces inside the crack or pit. As a result, the cracks or pits are darker than the remainder of the tool. This is in contrast with the polarization or oblique illumination, in which case cracks or pits are brighter than the remainder of the tool. The use of an extensive light source is important in providing a better image. When a relatively small or relatively collimated light source is used for imaging in reflection, the reflected light is quite intense when the tool surface is at an angle such that the reflected light is directed at the camera in the same way as the camera signal will be intense if the tool were replaced with a mirror and the mirror were reflecting the light directly onto the camera. With the extensive light source, the bright reflections are modulated because there are many angles that can reflect the light to the camera, but because the light source is large in size, the specular reflection is not so bright.

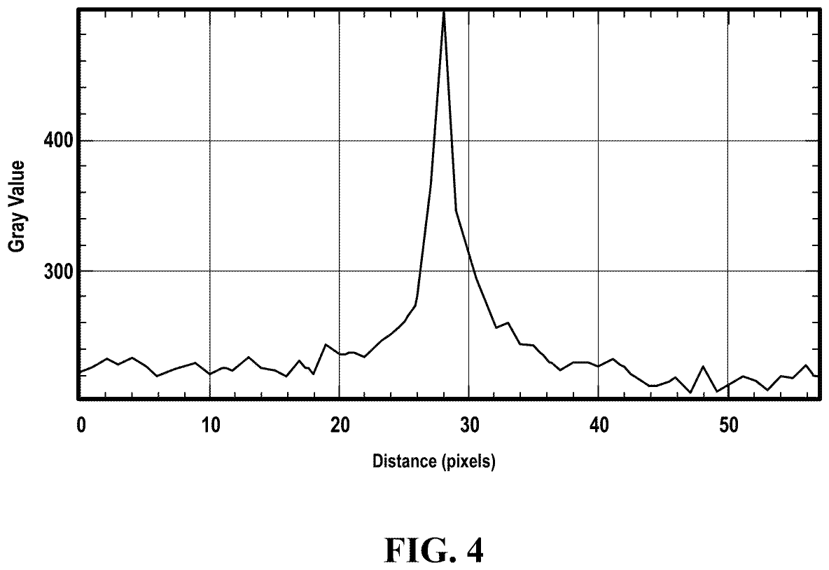

Polarization Imaging. Polarization imaging can provide contrast enhancement for cracks. A polarizer is an optical element that transmits light of a single polarization while blocking light at other polarizations. Polarization imaging uses polarization optical elements to enhance imaging based on the interaction of light polarization with the scene. The simplest form of polarization imaging uses crossed polarizers to block most of the light returning from the scene. For example, a vertically oriented polarizer might be placed in front of the illumination source and a horizontally oriented polarizer might be placed in front of the camera. Light that is directly retroreflected from the scene will retain the vertical polarization of the illumination and will in turn be blocked by the horizontal polarizer before reaching the camera. Because a crack can have steep walls that will be largely perpendicular to the tool surface, light that enters the crack is more likely to require multiple bounces or redirections before being retroreflected back toward the camera. These redirecting reflections will tend to scramble the polarization of the light, making the polarized light returning from the crack more likely to pass through the crossed polarizer in front of the camera. This can enhance the light returning from the crack relative to the light reflected by the tool surface. An example of polarization imaging enhancement of a crack is shown below and in FIG. 4. In the images below, a crack near the hinge is accentuated with polarization imaging. FIG. 4 shows a line for the crack in the instrument.

Below are images taken of surgical instruments with ambient light only (left) compared with polarization imaging (right). In both the top and bottom images produced by polarization imaging (right) the arrow points to defects that are not visible with ambient light only (left).

Oblique Illumination. Oblique illumination can enhance imaging of surface irregularities such as cracks, pits, and scratches as well as the sharpness of cutting edges. With oblique illumination, collimated light is used to illuminate the tool at a very small angle relative to the surface, while the camera is roughly orthogonal to both the illumination direction and the tool surface. In this configuration, the grazing angle of the light with respect to the surface produces only a small amount of light return from the tool surface. However, the surface irregularities such as cracks, pits, and scratches can produce specular reflections that result in strong signals on the camera. Specular reflections are those for which the incident and reflected light have the same angle relative to the surface normal. Specular reflections may be familiar as the bright glare from an icy road when looking towards the sun when it is low in the sky or the bright flashes of reflected sunlight on rough water that occur when the water surface happens to be at the appropriate angle. An example of enhanced detection of pitting using oblique illumination is shown below:

Below are images of a retractor taken with ambient light (left), with oblique illumination (middle), and with extensive light source in reflection (right). Comparing these three images, the images taken with the extensive light source in reflection and the oblique illumination show the pitting in the retractor more clearly than the image taken with ambient light illumination.

Below are images that show images of surgical instruments with biomass. The one on the left shows the surgical tool illuminated with conventional reflected light while the right image was from superimposed ultraviolet intrinsic fluorescence overlay on reflected light illumination.

Coverage and Throughput. Sensor Placement. Sensor placement should consider the need to inspect all sides of instruments. Options include: (a) Multiple sensors arranged around an instrument; (b) A combination of sensors and mirrors to enable visualization of all sides of an instrument; (c) An arrangement in which the instrument is rotated so that a single sensor is able to view the entire instrument (also possible by holding the instrument stationary and having the sensor move around the instrument); among other options.

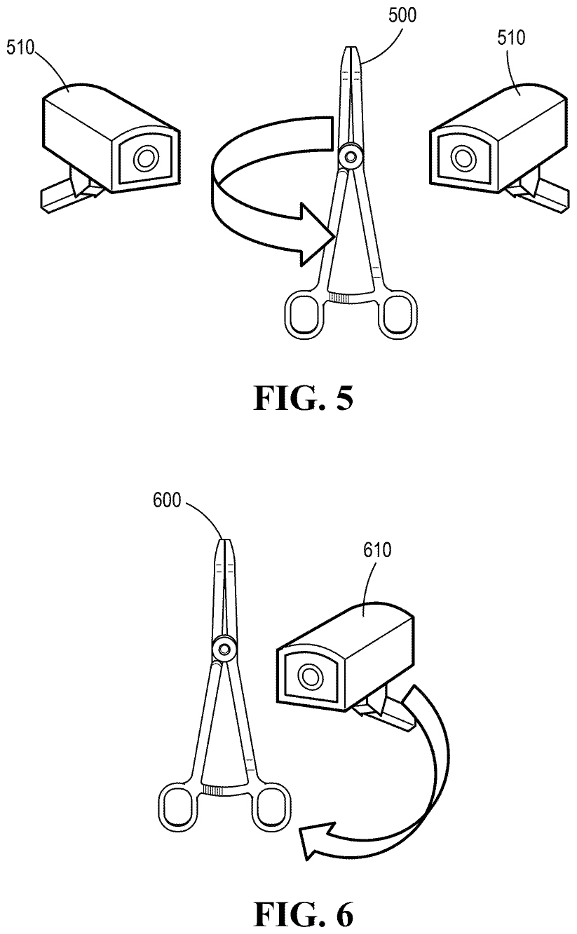

Sensor Movement. It is likely simpler to fix the positions of the sensors and move the instrument under examination, but both options could have potential advantages: Sensor(s) fixed in known locations and instrument presented to sensors (FIG. 5); Instrument could be rotated to allow all sides to be viewed; Multiple fixed sensors could be arranged such that fixed instrument is viewed from multiple sides simultaneously; One can use a single sensor and mirrors to take multiple angles simultaneously—For example, two mirrors can be placed behind the tool to acquire images from three sides (one direct image and two images from mirror reflections); Sensors could be mobile, on a fixed track with known position; Instrument is fixed and sensor(s) move about instrument (FIG. 6); Instrument and sensors are mobile (e.g., instrument rotates, sensors move on track to enable full coverage of instrument in reduced time) and Future sensors, especially for QR code readers, could be handheld.

Displays. Displays are likely to initially consist of images presented to operator for further review. With known instruments, display could highlight or otherwise indicate regions of interest or concern (e.g., hinges or areas of likely bioburden). False color displays could be used for UV, indicating areas of fluorescence (bioburden detection) and IR, highlighting damaged and pitted areas.

Analysis. Initial analysis will likely involve an operator making a decision based on the imagery presented from the sensors. Future versions could incorporate knowledge of the instrument and its history and potentially automated detection of damage or bioburden.

Automated Instrument Inspection & Handling System. The multimodal imaging system described above can form the central part of an automated instrument inspection and handling system. Both semi-automated and fully automated systems are possible. In the semi-automated system, certain tasks are implemented by a human, with automated processing and cues presented to the human to change pose, optimize orientation, and instructions for further steps such as spot cleaning or removal of a tool from use. For the automated system, robotic manipulation of the tool provides reliable control of the tool positioning and reorientation of the tool between imaging. The automation will make changing orientation between images more straightforward because the robotic mechanism (such as a robotic arm) can perform the proper manipulation of the tool between images while in synchronization with the camera acquisition, the timing of which is also under control from the same automated system.

Vision Augmented Inspection System. Computer vision methods can further improve the instrument inspection and handling. Computer vision can be used to (1) help with instrument identification, (2) help with assignment of bioburden or damage, or (3) track instrument wear over time.

Instrument Identification. The vision augmented inspection system will identify instruments based variously on Quick Response (QR) codes, radio frequency identification (RFID) tags, barcodes, or another unique device identification (UDI). This information can be augmented by basic instrument shape to provide redundancy or to help resolve instances whether the code or tag is partially obscured or cannot be read reliably for other reasons. Tool identification can be recovered by computationally comparing the tool with a database of 3D tool maps with automated or computational adjustment of the pose.

Assignment of Bioburden or Damage. The vision system can enhance the data from the multimodal imaging system. For example, at very small amounts of wear or damage or low levels of bioburden, there may be artifacts in the image due to scratches, nicks, markings, debris, or other variations. The vision system can help identify whether these smaller signals are actual wear/damage or bioburden by using the location of the blemish relative to different regions of the instrument. For example, for hinged instruments such as scissors and clamps, cracks tend to form near the hinge, and tend to radiate from the pin. Thus, blemishes in this region can more reliably be assigned as a crack, whereas a linear blemish in another region might be a scratch, and a compact blemish might be a nick. Similarly, biomass tends to accumulate inside joints; this information can help improve the accuracy of biomass detection.

Track Instrument Wear. The vision augmented inspection system will identify evidence of wear by location and depth as well as monitor the sharpness of cutting edges. This information will feed into the instrument data management system, where the data management system can track such information for each piece of instrument.

Instrument Data Management. A key part of the next generation SPD is instrument data management. This can be used to track information on instrument use and instrument wear. In some instances, the Instrument Data Management system can provide alerts to the user when an instrument's wear exceeds a certain threshold level of wear that has been predetermined.

Instrument Use Tracking. The next generation SPD will track each time an instrument is taken to the OR for use. Over time the types of use can be tracked to monitor instrument wear by procedure type, wear by different manufacturers for the same instrument type, wear by surgical team for the same procedure type. This information can be used to inform instrument purchases for longer lifetime, for advising manufacturers how their instruments can be improved, for inventory planning, and for understanding how to minimize costs.

Instrument Wear Tracking. The multimodal imaging system will track instrument wear and performance over time. This information may be used to understand how initial damage worsens over time, whether the propensity for accumulation of bioburden increases over time (if, for example, a sub-detectable amount of bioburden is established, that subsequently grows to a detectable level). Such information has value to instrument manufacturers to improve their products and to the SPD to improve their process and lower costs.

It may be appreciated by one of ordinary skill in the art, after being apprised of the present disclosure, that many other ways to configure a detection system are possible.

Other variations and modifications are possible. The description and illustrations are by way of example only. While the description above makes reference to various embodiments, it should be understood that many changes and modifications can be made without departing from the scope of the disclosure. Many more embodiments and implementations are possible within the scope of this invention and will be apparent to those of ordinary skill in the art. It is intended that the appended claims cover such changes and modifications that fall within the spirit, scope and equivalents of the invention. The invention is not to be restricted to the specific details, representative embodiments, and illustrated except in light as necessitated by the accompanying claims and their equivalents.

What is claimed is:

1. An apparatus for visually inspecting a surgical instrument in order to confirm its suitability for use, said apparatus comprising:
at least one light source for illuminating a sample;
at least one high resolution sensor for visually capturing characteristics of the sample;
whereby, when in use, the light emitted from the light source allows the sensor to capture details of the sample not easily discernible in the absence of the light.

2. The apparatus of claim 1, wherein sample is a surgical instrument.

3. The apparatus of claim 2, wherein the surgical instrument is selected from the group comprising cutting and dissecting surgical instruments; grasping and handling surgical instruments; clamping and occluding surgical instruments; retracting and exposing instruments; instruments for improving visualization; suturing and stapling surgical instruments; and suctioning and aspiration instruments.

4. The apparatus of claim 1, the light source is selected from the group consisting of incandescent, luminescent and reflective light sources.

5. The apparatus of claim 4, wherein the light source is reflective.

6. The apparatus of claim 5, wherein the light source is a mirror.

7. The apparatus of claim 1, wherein the sensor is an electronic image sensor.

8. The apparatus of claim 1, further comprising a filter for selectively restricting light waves.

9. A method of visually inspecting a surgical instrument comprising the steps of:
providing an apparatus for visually inspecting a surgical instrument comprising: at least one light source for illuminating a sample and at least one high resolution sensor for visually capturing characteristics of the sample;
providing a filter; and
altering the filter orientation.
whereby, when in use, the light emitted from the light source allows the sensor to capture details of the sample not easily discernible in the absence of the light.

10. The method of claim 9, wherein the sample is rotated so the sensor can capture multiple views of the sample.

11. An asset management method of visually identifying a surgical instrument comprising the steps of:
providing an apparatus for visually inspecting a surgical instrument comprising: at least one light source for illuminating a sample and at least one high resolution sensor for visually capturing characteristics of the sample;
illuminating the sample with the light source;
focusing the sensor on the sample.

12. The method of claim 11, wherein the focus characteristic of the sample is a unique identifier.

13. The method of claim 12, wherein the unique identifier is a Quick Response code.

14. The method of claim 12, wherein the unique identifier is a Radio Frequency Identifier.

15. The method of claim 11, wherein the light source of the apparatus is ultraviolet fluorescence.

16. The method of claim 11, wherein the light source of the apparatus is directed toward the sample by brightfield illumination.

17. The method of claim 11, wherein the light source of the apparatus is directed toward the sample by oblique illumination.

18. The method of claim 11, further comprising the step of logging indicia each time an inspection occurs.

19. The method of claim 18, wherein the indicia is selected from the group of time, date, inspector, light source, sensor type, inspection results and combinations thereof.

20. The method of claim 11, wherein sensor comprises machine vision technology.

21. The method of claim 11, at least one step is automated.
